(19) United States
(12) Patent Application Publication
(10) Pub. No.: US 2007/0087027 A1
(43) Pub. Date: Apr. 19, 2007

(54) Electrospun Skin Capable Of Controlling Drug Release Rates And Method

(76) Inventors: Skott E. Greenhalgh, Perkasie, PA (US); Rob Kiefer, Telford, PA (US)

Correspondence Address: INSKEEP INTELLECTUAL PROPERTY GROUP, INC, 2281 W. 190TH STREET, SUITE 200, TORRANCE, CA, 90504, US

(21) Appl. No.: 11/558,845

(22) Filed: Nov. 10, 2006

Related U.S. Application Data
(63) Continuation of application No. 10/313,496, filed on Dec. 6, 2002, now abandoned.
(60) Provisional application No. 60/372,721, filed on Apr. 11, 2002.

(51) Int. Cl. B32B 27/04; B32B 5/02; A61K 9/70

(52) U.S. Cl. 424/423; 442/123; 442/189; 442/239; 424/443

(57) ABSTRACT

A versatile covering process enabled through the identification and manipulation of a plurality of variables present in the electrospinning method of the present invention. By manipulating and controlling various identified variables, it is possible to use electrospinning to predictably produce thin materials having desirable characteristics. The fibers created by the electrospinning process have diameters averaging less than 100 micrometers. Proper manipulation of the identified variables ensures that these fibers are still wet upon contacting a target surface, thereby adhering with each other to form a cloth-like material and, if desired, adhering to the target surface to form a covering thereon. The extremely small size of these fibers, and the resulting interstices therebetween, provides an effective vehicle for drug and radiation delivery, and forms an effective membrane for use in fuel cells.

### CROSS REFERENCE TO RELATED APPLICATIONS

This application is related to U.S. provisional application Ser. No. 60/372,721 filed Apr. 11, 2002 and claims priority therefrom.

### BACKGROUND OF THE INVENTION

The process of the present invention yields a fabric and/or a fabric-like covering having multiple uses, and is particularly suited to medical device and industrial filtration applications. The covering may be created to have a wide range of desired characteristics, depending upon the intended application. The process generally involves electrospinning techniques.

Electrostatic spinning, or “electrospinning” is a process for creating fine polymer fibers using an electrically charged solution that is driven from a source to a target with an electrical field. Using an electric field to draw the positively charged solution results in a jet of solution from the orifice of the source container to the grounded target. The jet forms a cone shape, called a Taylor cone, as it travels from the orifice. Typically, as the distance from the orifice increases, the cone becomes stretched until, near the target, the jet splits or splays into many fibers prior to reaching the target. Also prior to reaching the target, and depending on many variables, including target distance, charge, solution viscosity, temperature, solvent volatility, polymer flow rate, and others, the fibers begin to dry. These fibers are extremely thin, typically measured in nanometers. The collection of these fibers on the target, assuming the solution is controlled to ensure the fibers are still wet enough to adhere to each other when reaching the target, form a randomly-oriented fibrous material with extremely high porosity and surface area, and a very small average pore size.

FIG. 1 is a diagram of the basic components required for solvent electrospinning. A polymer is mixed with a solvent to form a solution 1 having desired qualities. The solution is loaded into a syringe-like container 2 that is fluidly connected to a blunt needle 3 to form a spinneret 12. The needle 3 has a distal opening 4 through which the solution 1 is ejected by a controlled force 5, represented here in a simplified manner as being supplied by a plunger 6 but can be any appropriate controllable variable rate fluid displacement system and should be automated to ensure accurate flow rates.

A significant electric potential 7 is established across the spinneret 12 and a receiving plate 8. The electric potential 7 aids the force 5 in motivating the solution and by reducing the surface tension of the displaced polymer solution 1 from the spinneret 3 to the receiving plate 8. The combined action of the electric potential 7 and the displacement force 5 creates a jet of solution 9 that, due to the charge, splays at a position 10 between the spinneret 3 and the receiving plate 8. The splaying action creates a plurality of tiny threads or fibrils 11 that may or may not be dry upon reaching the plate 8, depending on the volatility of the solvent.

Electrospinning was first introduced in U.S. Pat. No. 1,975,504, which issued to Anton Formhals of Germany on Oct. 2, 1934. Formhals concentrated his efforts on using an electrical field in combination with a movable spool collection device to create a supply of relatively parallel, silk-like threads. Subsequent efforts by Formhals, such as described in his U.S. Pat. No. 2,160,962, were directed toward increasing the distance between the solution feeding device and the collecting electrode such that the threads are completely dry when collected and, thus, do not stick to each other.

Electrospinning did not become a viable manufacturing method for decades following Formhals's efforts because it failed to yield sufficient quantities of material, the output was inconsistent and of low quality, and the technological needs were insufficient to drive serious development of the process. Recently, however, applications such as medical filters and device coverings, as well as non-medical filtration applications, have lead the applicant to further development of electrospinning processes.

Electrospinning is presently the only way to create fibers having diameters measured in nanometers. Until now, however, electrospinning as a manufacturing process has not been refined to a point where it can be used to produce predictable, repeatable fabric. Moreover, uses for the electrospun fabric, especially medical uses, have heretofore not been defined and exploited.

### SUMMARY OF THE INVENTION

The present invention provides an electrospinning process that is useable to create a desired fabric with regularity. By manipulating appropriate variables, the electrospun fibers achieve characteristics that allow them to form a fabric that can adhere to an object, such as a stent, so that the object becomes covered; or the fibers can be used to create a free-standing fabric sheet or “skin” that has a variety of applications. FIG. 5 is a photograph of a stent covered with an electrospun fabric. Further, the skin may be stretched, orienting the fibrils of the skin into planes. Aligning the fibers results in increased tensile strength, altered permeability, reduced bulk, and reduced final part elongation (increased slope on the stress strain curve for the material). These stretching characteristics become very important when using the electrospun material to cover a stent. When the covered stent is deployed and expanded, the membrane cover is stretched radially, which in turn increases the membranes circumferential strength. The electrospun material comprises a plurality of randomly-oriented, inter-tangled, non-woven fibrils having an average diameter of less than 100 micrometers.

Thus, one aspect of the present invention provides a method for covering an object, such as a stent, with a fibrous polymer layer. The stent is covered with the fibrous polymer layer by providing a spinneret charged with an electric potential relative to a predetermined location on a target plate. The stent is placed between the spinneret and the predetermined location on said target plate. The polymer is then forced through the spinneret, thereby transferring at least some of the electric potential to the polymer such that the polymer forms a stream directed toward the target plate due to the electric potential between the liquid and the plate. Before it reaches the plate, the stream splays into a plurality of nanofibers due to the electric potential between the liquid and the plate. At least some, preferably most, of the nanofibers collide with the stent instead of reaching the target plate. The predetermined location on the target plate is then moved relative to the object until the entire object is covered. This is accomplished by moving the needle, electronically moving the point on the target plate where the potential is greatest relative to the needle, moving the object itself, or a combination of these three techniques.

Another aspect of the present invention includes a device and method for producing the device comprising an object, such as a stent, coated with a fibrous polymer. A distinction is now drawn between a covered object and a coated object. Especially applicable to objects that define a plurality of gaps, pores, or holes, such as stents, the distinction is based on how the polymer is distributed over the object. A polymer covered object, as used herein, is an object with a polymer that provides a somewhat continuous layer over substantially the entire outer surface of the object. The covering spans any gaps or holes defined by the object. Thus, a covered stent includes a polymer layer that spans the holes formed between the individual wires of the stent. FIG. 1 is an example of a covered stent.

A polymer coated object, as used herein, is one wherein the individual members that make up the object have a layer of polymer bonded to them. Thus, coated stents are made up of a plurality of woven wires that are each coated with a polymer, however, the gaps between the wires remain open. There are applications where coated stents are preferred over covered stents. However, the manufacture of coated stents has heretofore been accomplished by dip coating the stent in liquid polymer and allowing the stent to dry. This is problematic for numerous reasons. It is difficult to dip coat very small stents because the gaps between the individual wires become clogged with polymer due to the surface tension of the polymer solution. The polymer also tends to glue the individual wires of the stent together upon drying. When the stent later expands, the dried polymer coating cracks and flakes, causing a potentially dangerous situation whereby flakes of polymer enter the blood stream.

Thus, the coating method of the present invention begins with a covered object, preferably a stent, and heats the stent to a point where the fibrous, preferably electrospun, polymer loses its ability to span the gaps. The fibers spanning the gaps break and retract to the nearest wire by virtue of surface tension. The individual wires of the stent are now coated. The coating differs from that of a dip coating stent because, depending on the degree to which the stent was heated, the coating maintains a fibrous quality. The coating also typically only coats a fraction of the circumference of the wire. Thus, the fibrous coating is resistant to cracking and does not adhere the individual wires together. Expanding a coated stent of the present invention is analogous to rubbing two pipe cleaners together as opposed to breaking apart two wires that have been painted together.

One aspect of the present invention includes a method of using the electrospun fabric to deliver a drug to a target site. Applications for an electrospun fabric having a drug delivery capability include local topical delivery, antibiotics, orthopedic, cardiovascular, gynecological, hernia, anti-adhesion applications. There are four preferred methods of incorporating drug delivery with the electrospinning technology: 1) mixing a drug with a polymer prior to spinning the mixture, 2) using two spinnerets to spin a polymer and a drug separately and simultaneously, 3) impregnating a spun polymer with a drug, and 4) impregnating a spun polymer with drug-containing microspheres.

Using the first preferred method, a drug is mixed with the liquid polymers used in the spinning process. Electrospinning the resulting mixture yields fibers that contain the desired drugs. This method may be particularly suited to creating fibrils are not susceptible to being rejected by the body. Additionally, the fibrils can later be melted, compressed, or otherwise manipulated, thereby changing or eliminating the interstices between the fibers, without reducing the drug content of the fibrils.

Using the second preferred method, two spinnerets are used in close proximity to each other, each having a common target. One spinneret is loaded with a polymer while the other is loaded with a drug solution. The spinnerets are charged and their solutions are spun simultaneously at the common target, creating a material that includes polymer fibrils and drug fibrils. The drug being fed into the second spinneret may also be mixed with a second polymer to improve the spin characteristics of the drug.

The third method of drug delivery of the present invention involves impregnating an electrospun fabric with a drug. Some drugs may not be able to survive the electrospinning process. Taking advantage of the extremely small fiber sizes of electrospun fabric, and the correspondingly small size of the interstices between the fibers, allows the fabric to be impregnated with a liquid drug. For example, a polyester, such as PET, preferably spun over a scrim, may be impregnated with rapamycin. When spinning PET, hexaflouro-iso-propanol (HFIP), a volatile substance, is used to dissolve the PET into solution. Impregnating, such as by dip coating, the spun PET with rapamycin instead of mixing the rapamycin with the PET before spinning takes place, prevents the rapamycin from being destroyed by the HFIP. Mixing the rapamycin with a solution of PGA and PCL helps retain the rapamycin within the electrospun membrane.

A slow drug release effect may be obtained by impregnating the electrospun fabric with microspheres containing a desired drug. The microspheres further protect the drug from the manufacturing processes and from evaporation. Using the fourth method of drug delivery, the microspheres containing the drug are trapped within the interstices of the fabric and slowly dissolve in vivo, releasing the contained drug. An example of the polymers used to create a microsphere composite are a PCL membrane doped or loaded with a PGA microsphere form Alkermies. The polymer is preferably spun over a scrim. Polymer selection is important because the polymer used in the spinning process will define the drug release rates, material strength, stiffness, degradation times, and the like. Polymer selection also effects fabric elasticity. Polymers such as polyurethane, PGA, PLA, and PDO, create curly fibrils when spun. These curled fibrils behave like intertwined springs, thereby giving the fabric elastic qualities. FIG. 6 is a photograph, taken through a microscope, of an electrospun fabric having elastic qualities. Comparison may be made to Exhibits 3-6 which are photographs of non-elastic electrospun fabrics.

Drug release rates from a fabric are dependent on the difference in drug concentration between the fabric and the recipient tissue. As the drug is released, the concentration in the fabric drops while the concentration in the recipient tissue increases and later gradually decreases as blood carries some of the drug away. Thus, drug release rates are dynamic and can be collectively referred to as “drug release kinetics.” Drug release kinetics from a drug-containing fabric, such as an electrospun fabric, can be controlled further using a non-drug-containing, electrospun “cover”. The cover provides a barrier between the drug-containing fabric and the recipient tissue. Having a smaller average fibril size and smaller interstices than the drug-containing fabric allows the covering to restrict the drug release to a desired rate and effect low level drug release over an extended period. The cover can be made using the same or different polymer as the drug-containing polymer. Reference is made to FIGS. 7 and 8 that are microscopic photographs of an electrospun fabric having relatively large fibrils (on the order of 5 micrometers in diameter) that may be used as a drug-containing fabric. FIGS. 9 and 10 are microscopic photographs of an electrospun fabric having comparatively small fibrils (on the order of 1 micrometer in diameter) that may be used as a non-drug-containing covering.

A preferred application for a drug-containing fabric of the present invention pertains to a method of preventing intimal hyperplasia. Intimal hyperplasia is a medical condition whereby smooth muscle cells are directed to a damaged site in the interior of a blood vessel. The smooth muscle cells flock to the site to provide material for repairs in the form of scar tissue. Intimal hyperplasia can be a dangerous condition because it causes partial or complete blockage of the blood vessel. It has been found that intimal hyperplasia can be reduced by applying immunosuppressants to the damaged site. Conventional wisdom dictated that the drugs be introduced on the inside of the vessel, as close to the damaged site as possible. This has given rise to recent increased focus on the development of medicated stents and grafts. Medicated stents are an excellent mechanism for the direct application of a drug to the intima of a blood vessel. A discussion of medicated stents used to prevent intimal hyperplasia can be found in published U.S. Patent Application 20020143385 A1 to Yang, incorporated by reference in its entirety herein. A discussion of a graft designed to prevent intimal hyperplasia is discussed in U.S. Pat. No. 6,440,166 to Kolluri, incorporated by reference in its entirety herein. Kolluri focuses on the luminal wall of the graft to prevent intimal hyperplasia.

Surprisingly, medicated stents and grafts produced less-than-expected results when used to prevent intimal hyperplasia. Further research has found that sometimes the smooth muscle cells are accessing the target site by entering the vessel wall from the outside and traveling through the wall radially to the damaged site. Thus, contact with the medicated stent isn't made by all of the smooth muscle cells, just those that travel to the inner surface of the intimal tissue. Thus, a drug delivery mechanism that causes the smooth muscle cells to come into contact with an immunosuppressant before penetrating the vessel wall would be more effective at preventing intimal hyperplasia than a medicated stent placed in the lumen of the vessel. The free-standing electrospun fabric of the present invention, impregnated with an immunosuppressant, growth factor, cytokine, or other therapeutic agent, is an optimal drug delivery vehicle for this application.

The method for preventing intimal hyperplasia of the present invention, thus, includes wrapping a layer of electrospun fabric around the outside surface of a damaged or repaired vessel as a last step prior to closing the entry incision. The drug delivering wrap may be made of a degradable or non-degradable polymer. The wrap may be cut to size from a larger swatch of material prior to insertion. Preferably, the wrap is sized to completely cover the target site with an overlap on either side of the target site proportional to the degree of damage to be healed.

The present invention also includes application for the delivery of radiation to a patient. Radioactive material is expensive and difficult to safely handle and maintain. Further, radioactive decay creates complicated stocking issues. The electrospun fabric of the present invention may include a non-radioactive material that can later be “charged” with radiation. The material is introduced into the fabric by either mixing the material with the liquid polymer solution or impregnating the material into the interstices of the formed electrospun fabric. The material is preferably ¹⁶⁹thulium oxide, an isotope precursor, and becomes radioactive after it is “charged” by exposing it to radiation. Using a chargeable material, and waiting until just prior to insertion charge the material, allows the fabric to be produced, stored, and handled without the expense and safety concerns that typically accompany radioactive material.

Alternatively, a material such as calcium chloride or calcium phosphate may similarly be incorporated into the electrospinning process. These materials are characterized by attracting, rather than storing, radiation. Thus, a medical device is created that acts as a radiation target when implanted. The device focuses radiation on a desired location, thereby concentrating the radiation while protecting surrounding tissue. The result is a more efficient use of radioactive energy. Smaller doses may be used to achieve results that previously required stronger beams, less focused, beams that inevitably caused collateral damage.

Another aspect of the present invention provides a process for making a reinforced electrospun material with a scrim. The scrim is placed into the spinning chamber of the electrospinning apparatus and a polymer layer is electrospun directly onto the surface of the fabric scrim. This is advantageous because it incorporates the small fiber size of the electrospun material with the strength of the fabric scrim. Various techniques have been developed to improve the bond strength between the scrim and a spun membrane. The spun material can spun “wet” directly to the scrim cloth. The wet fibrils will stick to the scrim. The scrim cloth can be precoated with a thinned mixture of the spun polymer. This technique creates a sticky surface onto which fibrils may be spun.

Another aspect of the present invention provides a process for making a textured electrospun material with a scrim. The texturing process takes advantage of the wet, freshly electrospun polymer by stamping or rolling a texture into the polymer before the polymer is allowed to cure. Alternatively, a texture may be imparted to the fabric by forming the fabric on a textured substrate such as a screen. Texturing increases the ability of the membrane to wick fluids, improves the flexibility of the material, allows the material to drape better, and reduces material stiffness. The texturing process can be performed on dry membranes by using either heat or solvent to soften the membranes.

Still another aspect of the present invention provides a process for using an electrospun polymer layer as an adhesive to bind a previously-spun polymer cover to an object or substrate. The wet polymer used is preferably identical to the previously-spun polymer covering. There are several advantages to using a wet, electrospun polymer layer instead of an adhesive, for this purpose. First, the glue and fabric are identical, reducing the chance for bond failure. Second, material requirements are reduced as well as material handling complications. For example, most glues, such as PMMA, cyanoacrylate, epoxies, and the like, are toxic. Use of these adhesives for medical applications adds significant complications and safety considerations. Third, no heat is necessary to bind the previously-spun material to the binding polymer layer. Adhesives often require heat, which may weaken the fabric. Fourth, using the same polymer as a binding agent that was used to make the fabric results in a device comprised of fewer types of materials so, potentially, the regulatory path for a medical product may be shortened.

Yet another aspect of the present invention is a process for electrospinning a composite material. Composite materials include more than one electrospun polymer and combine the distinct advantageous of each polymer. The process may include mixing the polymers into one spinneret or using two spinnerets and spinning the materials from each simultaneously onto a common target.

Still another aspect of the present invention provides a process for electrospinning a composite material useable for fuel cells. Fuel cells work by separating two electrodes with a polymer membrane designed to inhibit certain charged atoms. The membranes are typically manufactured with zero porosity using a perfluorosulfonate ionomer sold by Dupont called Nafion®. In some cases the membrane is reinforced with scrim fabrics in a laminating process. Using the impregnating techniques described above, electrospun material may be impregnated with a Nafion®-like material to manufacture membranes with improved conducting performance, strength, durability, and most importantly, reduced manufacturing costs.

Additionally, the surface area of an electrospun membrane will affect the transpor/cell efficiency. By using a bulky membrane containing Nafion®, polymer surface area may be drastically increased without significantly increasing membrane thickness. Also, membranes having thicknesses that vary across the extents of the membrane can be manufactured by manipulating polymer flow rate, fibril size, temperature, or pressure, so long as the cell has a section of zero porosity.

Preferably, the fuel cells are made using 100% Nafion® mixed at a ten to one ratio with polyethylene oxide to create the fibril solution for spinning. Polyethylene oxide is used to thicken the Nafion® polymer. Alternatively, composite membranes may be by spinning Nafion in one spinneret and PET, PP, PU from a second spinneret. Additionally, Nafion® may be spun directly onto both sides of an open scrim cloth of a material such as PET, PTFE, PP, or PEEK, using heat and minimal pressure to attain the desired texture or bulk of the membrane.

### BRIEF DESCRIPTION OF THE DRAWINGS

FIG. 1 is a diagrammatic representation of the basic components of a known electrospinning apparatus;

FIG. 2 is a perspective view of a preferred electrospinning apparatus of the present invention; and,

FIG. 3 is a perspective view of the power supply of a preferred embodiment of the present invention;

FIG. 4a is a perspective view of a pump of a preferred embodiment of the present invention;

FIG. 4b is a perspective view of a pump of an alternative embodiment of the present invention;

FIG. 5 is a photograph of a covered stent of the present invention;

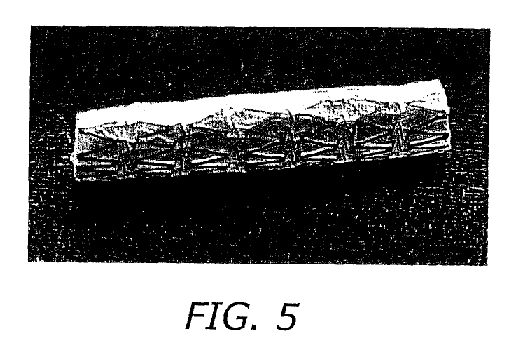

FIG. 6 is a photograph of an elastic electrospun fabric of the present invention;

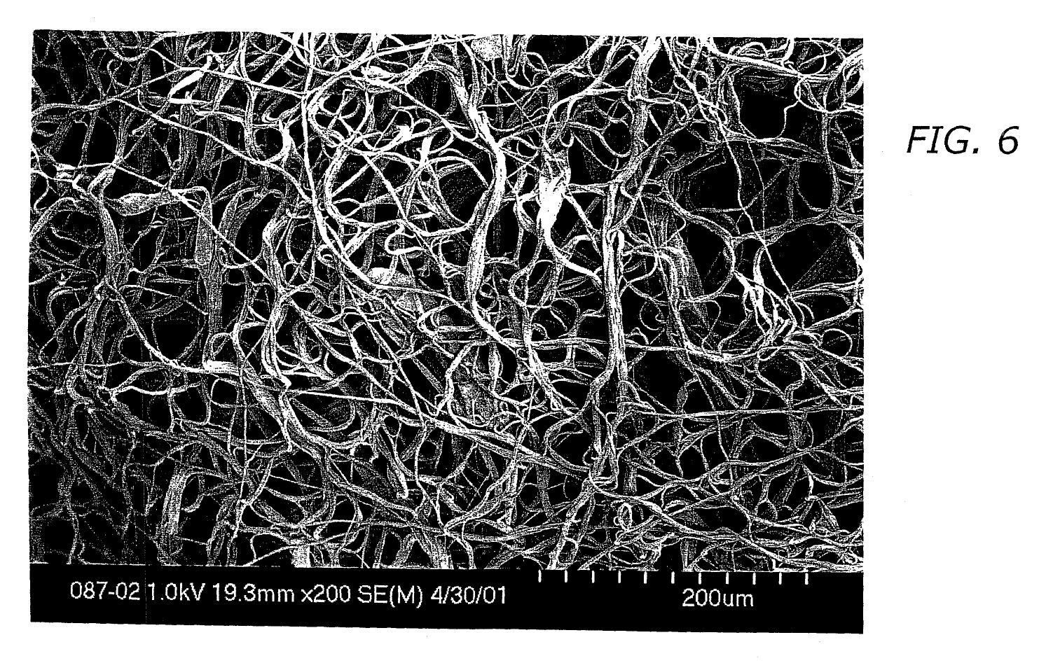

FIG. 7 is a photograph of an electrospun fabric of the present invention having relatively large fibrils;

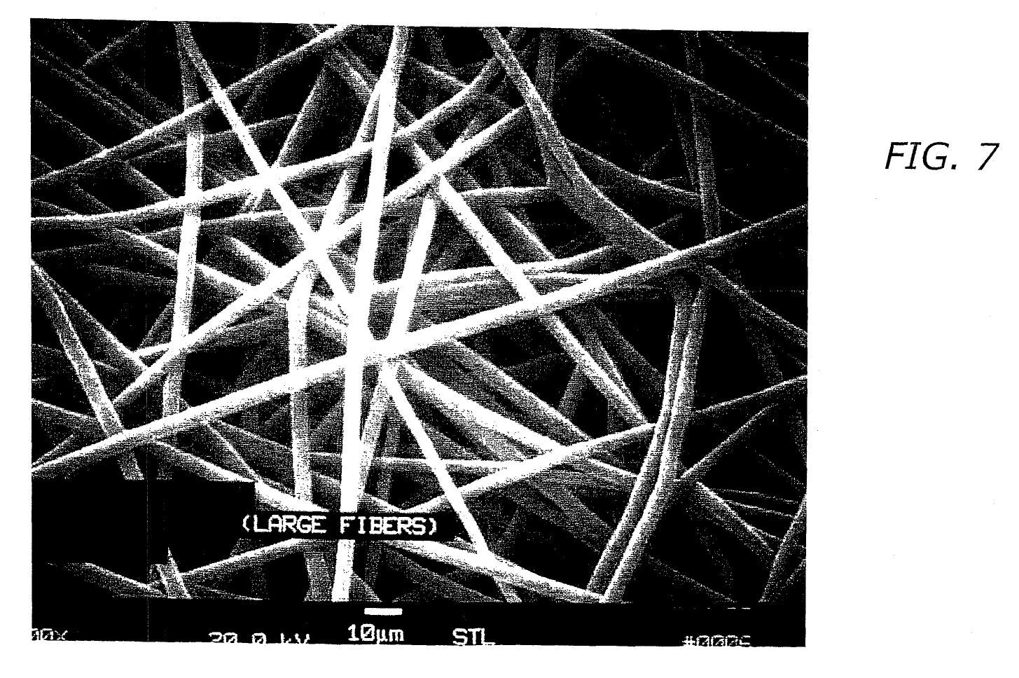

FIG. 8 is a photograph of the electrospun fabric of FIG. 7 taken at an edge of the fabric;

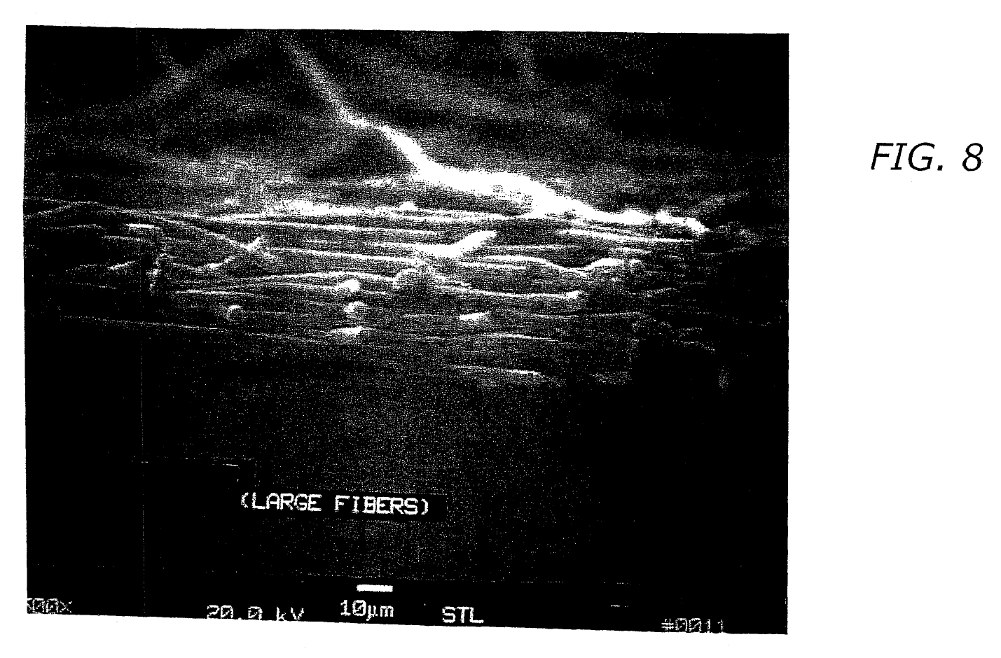

FIG. 9 is a photograph of an electrospun fabric of the present invention having relatively small fibrils when compared against the fibrils of the electrospun fabric of FIG. 7; and

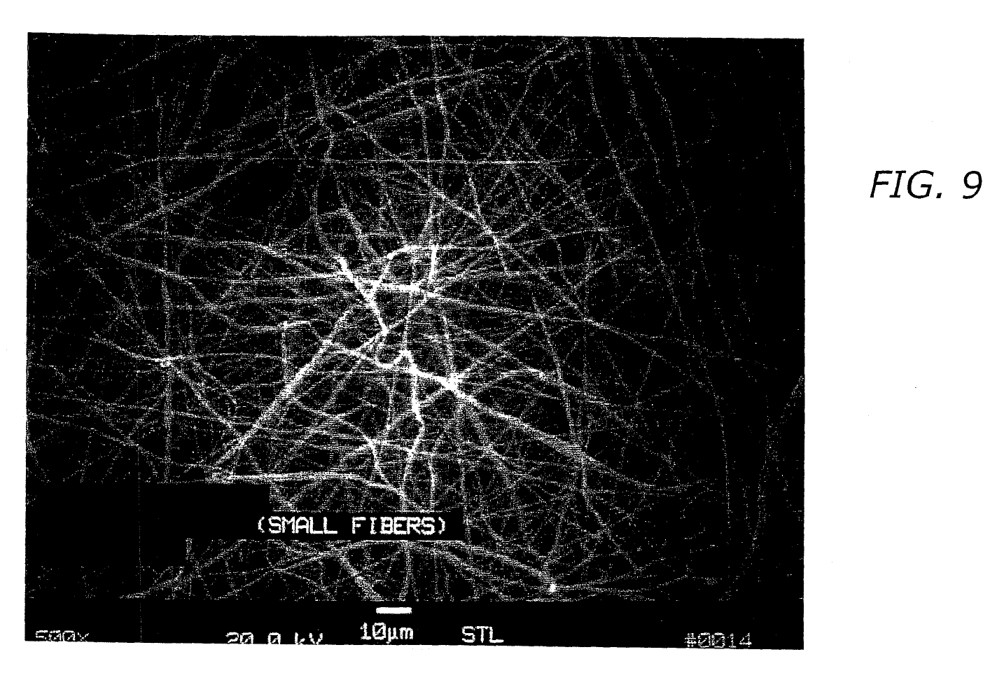

FIG. 10 is a photograph of the electrospun fabric of FIG. 9 taken at an edge of the fabric.

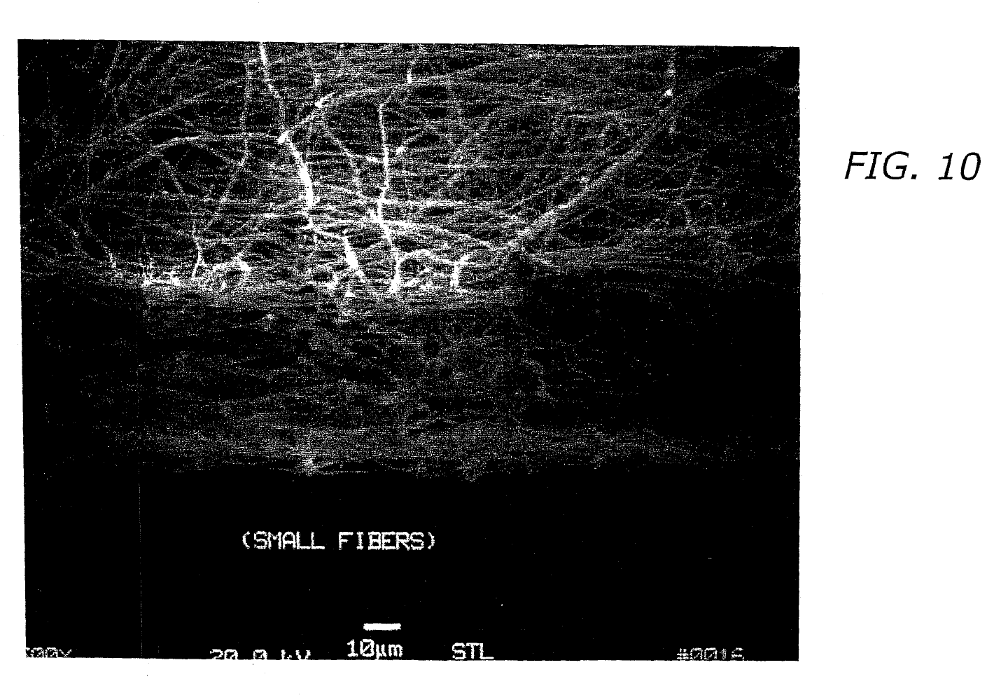

### DETAILED DESCRIPTION OF THE INVENTION

Mechanical Setup of the Electrospinning Process of the Present Invention

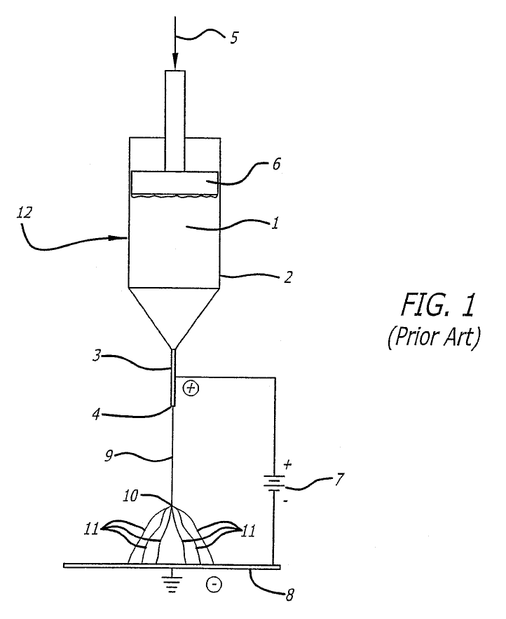

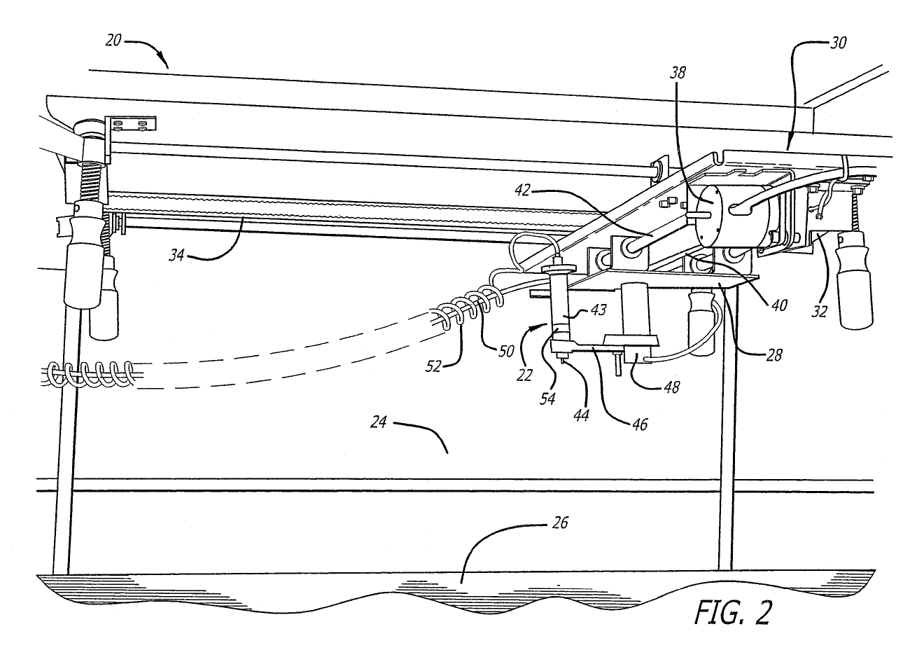

Referring now to FIG. 2, there is shown a preferred mechanical setup of the electrospinning process of the present invention. Though some of the components are similar to those of FIG. 1, all components have been given new numbers for purposes of clarity.

The electrospinning apparatus 20 includes a spinneret 22 over a spinning chamber 24, which is defined at its lower end by a collection plate 26. The spinneret 22 is mounted to the carriage 28 of an x-y translator 30, which is preferably an electronically controlled motion system. The x-y translator 30 relocates the spinneret 22 anywhere within the spinning chamber 24 in a horizontal plane at the top of the chamber.

The x-y translator 30 includes an x motor 32 that is operably attached to a first belt 34 for translating the carriage 28 along a pair of first horizontal guide bars 36. The translator 30 also includes a y motor 38 that is operably attached to a second belt 40 for translating the carriage 28 along a pair of second horizontal guide bars 42 that are perpendicular to the first horizontal guide bars 36.

The spinneret 22 includes a syringe 43 and a needle 44. The needle 44 may be of varying sizes but, for most applications, is optimally a 20 gauge needle. The spinneret is mounted to the carriage assembly 28 with an adjustable bracket 46, which also acts as an electrical connection point for the positive DC power. The bracket 46 connects to a mounting post 48 and is constructed and arranged so that it may be relocated up or down the mounting post 48, thereby providing a height adjustment for the spinneret 22. The mounting post 48 is also an electrical insulator, isolating the positive DC power from the rest of the apparatus 20.

The spinneret 22 is also connected to a positive DC power cable 50 and a pressure line 52. The power cable 50 provides the necessary positive DC potential to affect the electrospinning process. The pressure line 52 allows remote control of the syringe 43. The pressure line 52 carries fluid under pressure that is used to put downward force on the plunger 54 of the syringe 43. The fluid is preferably compressed air or nitrogen, but may be any compressible, or non-compressible fluid.

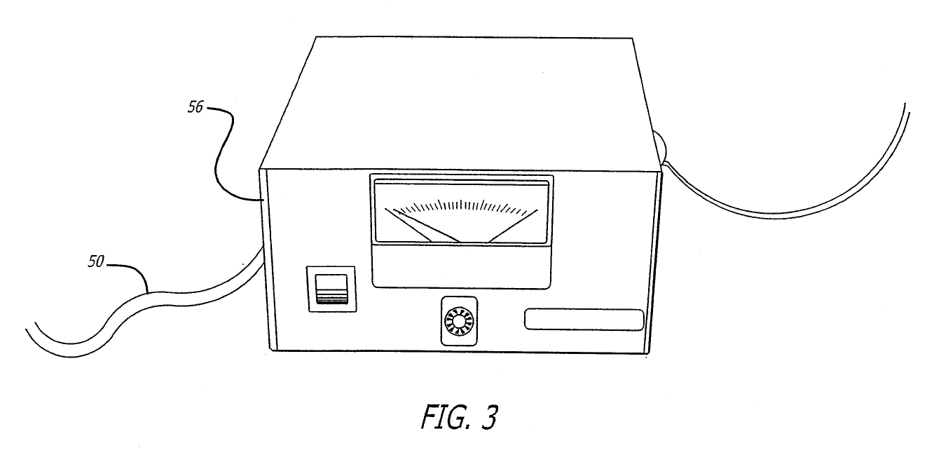

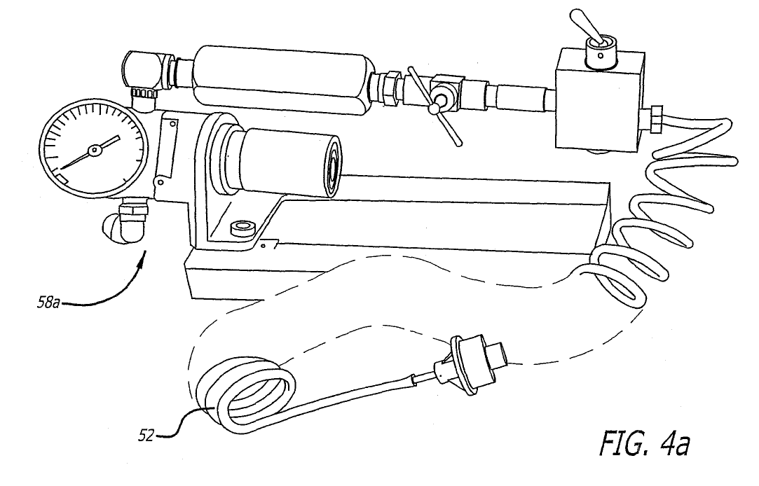

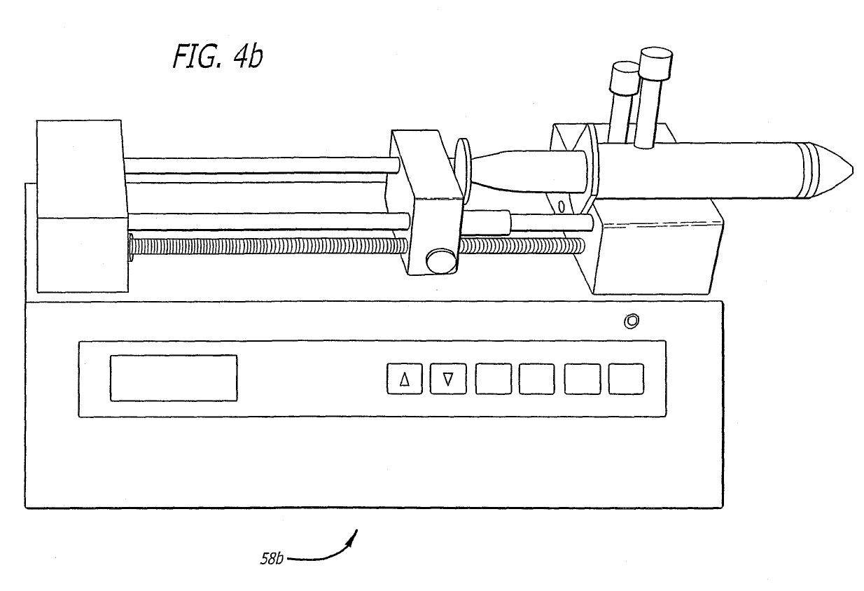

Reference is now made to FIG. 3-4. Preferably outside of the spinning chamber 24, the power cable 50 and the pressure line 52 are connected to a power supply 56 (FIG. 3) and a pump 58 (FIGS. 4a and 4b), respectively. The power supply 56 preferably provides between 0 and 30 kV DC. A preferred pump 58a, shown in FIG. 4a, uses compressed air or nitrogen from a domestic source to apply pressure through the coiled pressure line 52 to the syringe 43. An alternative pump 58b, shown in FIG. 4b, is a syringe pump, such as those made by Harvard Apparatus, and also functions to apply pressure through the coiled pressure line 52 to the syringe 43. Both pumps 58 have adjustable feed rates.

A computer (not shown) is preferably in data flow communication, and thus controls, both of the motors 32 and 38, the power supply 56 and the pump 58. The computer executes various task-specific programs that provide optimal control over many of the variables inherent in the electrospinning process. A preferred controller program for the X-Y translator motors 32 and 38 is MD2 commercially available from Arrick Robotics of Hurst, Tex. A computer program for controlling the pump and providing direction to the MD2 program has been developed. The program, however, is little more than a memory device, for storing parameters for a given desired fabric output, and executing commands that are input directly before a spinning process begins.

Identified Variables

The various aspects of the present invention are facilitated by astute identification and manipulation of a significant number of variables. Understanding these variables, and their impact on the results of the electrospinning process allows the creation of fibrous materials having one or more of many different desired properties using electrospinning.

The variables that were identified in the present invention include:
1. Polymer type
2. Viscosity of the polymer
3. Conductivity of the polymer
4. Electric potential
5. Spinneret size
6. Distance to the collection area
7. Air temperature/humidity
8. Polymer feed rate
9. Relative motion between the spinneret and the collection area
10. Pressure in spin chamber
11. Chemical used to soluabilize the polymers
12. Polymer crystallinity

1. Polymer Type. The general requirements for a polymer to be used in the electrospinning process are that the polymer must flow and have cohesive properties to form fibers. Polymers having these characteristics form a group from which individual selections may be made based on the intended purpose of the electrospun material.

For example, it is often desired that temporary medical devices used in vivo degrade over time so removal surgery is not necessary. Thus, degrading polymers are chosen for these applications. Degrading polymers suitable for electrospinning include: Poly(L-lactide) (PLA), 75/25 Poly(DL-lactide-co-E-caprolactone), 25/75 Poly(DL-lactide-co-E-caprolactone), Poly(E-caprolactone) (PCL), collagen, Polyactive, and Polyglycolic acid (PGA). There are many acceptable volatile organic liquids usable to dissolve these polymers. Examples of these solvents include: hexafluoro-iso-propanol, dichloromethane, dimethylacetamide, chloroform, and dimethylformamide. The concentration of solute to solvent can have dramatic effects on the finished product. For example, lower solute concentration can result in a decreased production rate for a given number/size of spinnerets, smaller fiber diameters, lower permeability, and lower porosity

Other applications call for materials that do not degrade. Non-degrading polymers that are acceptable for electrospinning include: polytetrafluoroethylene, polyurethane, polyester, polypropylene, polyethylene, and silicone. Again, a volatile organic liquid, such as dimethylacetamide, methylene chloride, dimethylformamide, hexafluoro-iso-propanol for polyurethane, hexafluoro-iso-propanol for polyester and xylene at 90 C for polypropylene, should be chosen as a solvent.

2. Viscosity of the Polymer. Successful results are achieved using polymers having viscosities between 1 and 50 centipoise. Generally, polymers having higher viscosities generate larger fibers.

3. Conductivity of the Polymer. Changing the conductivity of the polymer inversely changes the size of the fibers. In other words, increasing the conductivity of the polymer, reduces the size of the resulting fibers. The polymer conductivity can be changed by adding an ionic material, such as salt, to the polymer solution.

4. Electric Potential. Increasing the electric potential between the spinneret and the receiving plate decreases the size of the electrospun fibers.

5. Spinneret size. Spinneret size determines the size of the polymer stream exiting the spinneret needle. If the stream is too large, the stream will splay later, or not at all, for a given voltage level. Splaying later, or closer to the target, results in a wetter deposit onto the target. The occurrence of unacceptably large fibrils also increases with spinneret size. Conversely, if the spinneret needle is too small, the stream may splay too soon and the fibrils will be dry upon reaching the target.

6. Distance to the Collection Area. The distance to the collection area most affects how wet the spun fibers will be when they hit the target. If the distance is shorter, the fibers will still be quit wet when they hit, increasing the degree to which they stick together and to the target. Thus, if it is desired to get fibrils to adhere to a substrate, the needle may be lowered. Conversely, if it is desired to create a thick, lofty material, the needle may be raised.

7. Air Temperature. As the air temperature increases, the needle height must decrease to maintain similar fiber drying behavior. Reducing the air temperature in the spinning chamber tends to make the fibrils wetter for a given spinneret height, as fiber drying rate is reduced.

8. Polymer Feed Rate. Increasing the flow rate of the polymer through the spinneret increases the loft of the membrane, increases stiffness, reduces the ability of the material to resist delamination, reduces adherence of the membrane to other substrates, and reduces the ability to trap materials within the membrane.

9. Collection area motion. The relative motion between the spinneret and the collection area affects several of the properties of the resulting material. If the surface of the target being covered is moving under the spinneret, but the spinneret is still relative to the conducting plate, such as would be the case if a stent were being rotated under a steady stream, as the speed of rotation is increased, the thickness of the resulting material will be reduced, and the fibers making up the material will tend to be more aligned with each other. This can affect the strength, stiffness and porosity of the resulting material. If the needle is moving relative to the conducting plate, thereby increasing the distance that the polymer stream is travelling, then the effects associated with changing the spinneret height emerge.

10. Pressure in spin chamber. Changing the atmospheric pressure in the spin chamber affects the drying rate of the spun polymer; lower pressure will accelerate the drying process, high pressure will retard the drying or solvent evaporation. Thus, if the fibrils are too dry or too wet when they strike the target surface, one way to adjust the drying rate is to adjust the pressure in the spin chamber.

11. Solvent used. Solvents that are more volatile, i.e., xylene, acetone, HFIP, and chloroform, tend to react better to spin chamber pressure changes.

12. Polymer crystallinity. Most polymers can be made to have lower crystallinity. Lower crystalline polymers react well to spin chamber pressure changes as amorphous regions in polymers release solvents faster than regions with higher crystallinity. Therefore, for an amorphous polymer, increased pressure can be used to accurately effect slower drying and better fibril bonding.

Process for Making a Drug Delivering Material

Now described is a preferred method of using the electrospinning technology to create a material that facilitates drug elution when the material is placed in vivo. A polymer-based solution is developed, preferably of a polymer, a solvent and an immunosuppressant.

A preferred polymer for this application is developed by mixing PolyDL-Lactide (PLA) at 15-20% by mass, preferably at 17.90% by mass with a solvent such as HFIP at 80-85% by mass, preferably at 82.10% by mass. A preferred immunosuppressant is then added at 0.05% of polymer mass. Preferred immunosuppressants include rapamycin, taxol, and warfin. This mixture is allowed to fully dissolve. Other acceptable polymers include, but are not limited to: polyester (PET), polyglycolide acid (PGA), polycaprolactone (PCL), polydioxanone (PDO), and polyurethane (PU). Preferably, if these other polymers are to be used, they are used at 10-20% by mass with a solvent such as HFIP at 80-90% by mass.

A substrate, such as a course mesh screen, is used as the target plate so that the material may be removed from the plate without damage. The screen is highly open and allows drying and curing from both sides. Furthermore, the limited surface area of the screen promotes an easy membrane release. In order to become the target plate, however, the substrate must conduct electricity so that it may be grounded. Grounding the substrate, such as by connecting it to a ground cable, is essential to establish the electric potential between the spinneret and the substrate. A stretchable material, such as a screen is preferable so when the substrate is stretched, the material separates from the substrate and is easily removed. Additionally air currents can be drawn through the screen that coalesce the spun polymer into more discrete spin patterns where the polymer has a higher density.

The motion controller and the computer of the electrospinning device are then energized and the computer program for the motion controller is initialized. A preferred controller program is MD2 commercially available from Arrick Robotics of Hurst, Tex. Prior to running the program, a predetermined quantity of the solution, preferably 4.0 mL, is transferred into the spinneret. The piston is inserted into the bore of the spinneret barrel and the barrel assembly is inverted. The piston is then depressed until all the air has been ejected from the barrel. A needle, preferably a 20 gauge needle, is then secured to the end of the barrel.

Alternatively, if a barrel-less system is used, the desired quantity of solution is programmed into the computer. The barrel-less system is a manifold based, multi-spinneret system. Each spinneret is connected to the manifold, which is fluidly connected to a feed reservoir. The feed rate of the solution is controllable through the use of pressurized fluid which is applied to the reservoir in order to control the rate of dispensation.

Next, the pump is connected to the spinneret assembly and the needle height is adjusted to a predetermined height, optimally 12.00″. The DC power supply is also energized to a predetermined value, which for this application, is optimally 19 kV.

The pump is energized and adjusted to a predetermined flow rate, preferably 0.60 mL/minute. If a syringe barrel system is being used, the pump mechanically moves the barrel through the syringe at a predetermined rate to control flow rate. If a barrel-less system is used, pressure is manipulated to control the flow rate through the spinneret.

The desired computer program is now run in order to obtain the appropriate fabric properties, such as thickness, areal density, dimensions. The computer program is a means of storing parameters for a given desired fabric output. The computer program directs the motion controller to make an appropriate number of passes until a desired material thickness or areal density is obtained.

After the program has run and stopped, the power supply and the pump are turned off and the substrate is removed from the spinning cavity, with the newly electrospun material remaining on the substrate. The material is allowed to cure before it is removed from the substrate. Preferably, for this application, the material is allowed to cure for at least three hours. The material is then removed from the substrate by gradually pulling on the corners of the screen until the material separates from the screen. This process can be accelerated using radiant or convection heat, preferably below the galss transition temperature of the spun polymer.

Next the material is rinsed in a cleaning solution, preferably de-ionized water, CO₂, methanol, alcohol, xylene, sterile water, or the like, for two minutes. The purpose of this rinsing step is to remove any surface drugs that may be present. Removing the surface drugs is desirable because the polymers are designed to deliver a therapeutic level of drug at a predetermined rate. The surface drugs, if not removed, would be delivered immediately at an uncontrolled rate and in addition to the intended dosage. The presence of surface drugs is due to leaching that occurs while the polymer and solvent are curing. The material is then allowed to dry.

Next the newly formed material is cut into pieces of a predetermined size and shape. The size and shape of the material is determined by customer request or, if packaged based on a use specific application, by intended use. Consideration is given to the amount of drugs per unit of area present in the material. Notably, because the drugs are delivered directly to the tissue contacting the material, the amount of drug necessary for a given application is extremely less than would be needed to accomplish a similar effect giving the drugs orally or via injection.

The material is now ready to be inspected and packaged. Inspection, at a minimum, tests one or more samples per “run” to determine properties of the material such as thickness, porosity, and areal density, tensile strength, suture retention and dug dosage using a chemical extraction. Optimal values for these properties vary widely with intended application. Some orthopedic applications require thicker, 0.01″ more porous membranes, greater than 100 micron pores, such as miniscal repairs. For most vascular applications, thinner (on the order of 0.002 inches) and less porous (below 300 cc/cm²/min with 50 micron pores) are suitable. One or more assays are also conducted to determine actual drug content. If acceptable, the other pieces are packed individually into separate containers. Pouches made of a lint-free material such as Tyvek®, made by DuPont®, adequately protect the pieces. Finally, the material and pouches are sterilized using ETO, gamma, ebeam, or the like.

Process for Making a Radiation Delivering Material

Electrospinning may be used to create a material that is capable of delivering a radioactive isotope to a target site in vivo. Beta emitting isotopes are preferred because beta radiation has a low penetration depth, ideal for applications where the source material is directly in contact with the target tissue. There are two preferred methods of making a material capable of delivering a radioactive isotope. The first method spins a “cold” isotope into the electrospun material. The material can then be made “hot” by subjecting the isotope-containing material to radiation. This method obviates the need for increased radiation precautions during manufacture. The second method spins a “hot” isotope into the electrospun material. Each method has distinct advantages.

The cold spinning process increases material shelf life because radioactive decay does not begin until the material is charged prior to usage, or after the material has been inserted into the body. However, isotope selection for this application is somewhat limited. Not all isotopes absorb radiation at the same rate. If the absorption time is too great, the polymer will degrade before the membrane is hot enough. Thus, hot spinning provides a way to take advantage of many more isotopes. Both manufacturing processes are relatively easy to perform using the advances of the present invention.

The first process, wherein a cold isotope is spun into the material, begins with making a solution of a polymer, a solvent and a precursor isotope. The polymer is preferably PLA at 17.90% by mass. The solvent is preferably HFIP at 82.10% by mass, and the precursor isotope is preferably ¹⁶⁹Thulium Oxide at 1% of polymer mass. Thulium compounds have an affinity to accept neutrons from bombardment in a nuclear reactor. The polymer and the solvent are mixed together, and then the isotope is added and allowed to fully dissolve.

A substrate, such as a course mesh screen, is used as the target plate so that the material may be removed from the plate without damage. In order to become the target plate, however, the substrate must conduct electricity so that it may be grounded. Grounding the substrate, such as by connecting it to a ground cable, is essential to establish the electric potential between the spinneret and the substrate. A stretchable material, such as a screen is preferable as a substrate so that when the substrate is stretched, the material separates from the substrate and is easily removed.

The motion controller and the computer of the electrospinning device are energized. The MD2 computer program for the motion controller is initialized. Prior to running the program, a predetermined quantity of the solution, preferably 3.2 mL, is transferred into the spinneret. If a barrel system is being used, the piston is inserted into the bore of the spinneret barrel, the barrel assembly is inverted, and the piston is depressed until all the air has been ejected from the barrel. A needle, preferably a 20 gauge needle, is then secured to the end of the barrel.

Alternatively, if a barrel-less system is used, the desired quantity of solution is programmed into the computer. The barrel-less system is a manifold based, multi-spinneret system. Each spinneret is connected to the manifold, which is fluidly connected to a feed reservoir. The feed rate of the solution is controllable through the use of pressurized fluid which is applied to the reservoir in order to control the rate of dispensation.

Next, the pump is connected to the spinneret assembly and the needle height is adjusted to a predetermined height, optimally 12.00″. The DC power supply is also energized to a predetermined value, which for this application is optimally 23 kV.

The pump is energized and adjusted to a predetermined flow rate, preferably 0.60 mL/minute. If a syringe barrel system is being used, the pump mechanically moves the barrel through the syringe at a predetermined rate to control flow rate. If a barrel-less system is used, pressure is manipulated to control the flow rate through the spinneret.

The desired computer program is now run in order to obtain the appropriate fabric properties, such as thickness, areal density, dimensions. The computer program is a means of storing parameters for a given desired fabric output. The computer program directs the motion controller to make an appropriate number of passes until a desired material thickness or areal density is obtained.

After the program has run and stopped, power supply and pump are turned off and the substrate is removed from the spinning cavity, with the newly electrospun material remaining on the substrate. The material is allowed to cure before it is removed from the substrate. Preferably, for this application, the material is allowed to cure for at least three hours. The material is then removed from the substrate by gradually pulling on the corners of the screen until the material separates from the screen.

Next the material is rinsed in a cleaning solution, preferably de-ionized water, CO₂, methanol, alcohol, xylene, sterile water, or the like, for two minutes. The purpose of this rinsing step is to remove any surface isotopes that may be present. Removing the surface isotopes is desirable because the polymers are designed to deliver a therapeutic level of isotopes at a predetermined rate. The surface isotopes, if not removed, would deliver radiation at an uncontrolled rate and in addition to the intended dosage. The presence of surface isotopes is due to leaching that occurs while the polymer and solvent are curing. Once the material is rinsed, it is allowed to dry.

Next the newly formed material is cut into pieces of a predetermined size and shape, preferably determined by customer requirements. Consideration is given to the desired dose per unit area and the time of dosage, which is defined by the half-life of the isotope.

The material is now ready to be inspected and packaged. Inspection, at a minimum, tests one or more samples per “run” to determine properties of the material such as thickness, porosity, and aerial density. Optimal values for each are dependent on the customer requirements. One or more assays are also conducted to determine actual drug content. If acceptable, the other pieces are packed individually into separate containers. Pouches made of a lint-free material such as Tyvek®, made by DuPont®, adequately protect the pieces.

The material and pouches are ready to be placed in a nuclear reactor to accept neutrons. The amount of radioactivity received is directly proportional to the amount of time spent in the reactor, and the energy levels in the reactor. The reactor used was the MITR-II, a tank-type reactor owned by the Massachusetts Institute of Technology (MIT). Preferably, the material is placed in the reactor for between 30 and 60 minutes, more preferably between 40 and 50 minutes, and the reactor power is set at preferably between 1 and 10 megawatts, more preferably between 3 and 7 megawatts. Positive results were obtained placing polyurethane doped with Thulium for 42 minutes at 5 megawatts. After the appropriate time has elapsed, the pouches containing the now radioactive material are subjected to an assay to determine actual energy level and then sterilized.

The second method of producing a radioactive material is virtually the same as the first, described above, with a few exceptions. The preferred isotope is ⁴⁵calcium chloride, which has the appropriate beta energy level, half-life, and is relatively harmless. The isotope is mixed with the solution as described above, with appropriate handling measures taken for working with radioactive material. The only other exception is that the material is not placed in a reactor after it is produced, as it is already radioactive.

Process for Making Membranes from High Melt Temperature Thermoplastics.

Now described is a preferred method of using the electrospinning technology to create a material that would usually require vast amounts of heat, greater than 600 F, to melt and extrude or spin into fibers. When the material is mixed with a solvent, however, it dissolves well below its melting point.

Thus, first a polymer-based solution is developed, preferably of a polymer, and a solvent. A preferred polymer solution for this application is developed by mixing a low crystalline polyetherimide, such as Ultem® made by General Electric Plastic®, at 18.00% by mass with a solvent, preferably chloroform, at 82.00% by mass. This mixture is allowed to fully dissolve. Other acceptable polymers for this application include PEEK, PTFE, PEK, ETFE, and pitch carbon graphite. If it is desired to use less crystalline polymers such as PU or Ultem, or highly volatile solvents such as chloroform, xylene, HFIP, or acetone, extra steps are taken to ensure the fibrils will be wet when they hit the target. These steps may involve changing the atmosphere in the spinning chamber by increasing the pressure therein or lowering the chamber temperature. Alternatively, the solvents may be mixed to make them less volatile.

A course mesh screen, preferably aluminum, is placed into the bottom of the spinning cavity for use as a substrate. The substrate will eventually be connected to the positively charged cable.

The motion controller and the computer of the electrospinning device are energized. The MD2 computer program for the motion controller is initialized. Prior to running the program, a predetermined quantity of the solution, dependent on the amount of material to be produced, is transferred into the spinneret. If a barrel system is being used, the piston is inserted into the bore of the spinneret barrel, the barrel assembly is inverted, and the piston is depressed until all the air has been ejected from the barrel. A needle, preferably a 20 gauge needle, is then secured to the end of the barrel.

Alternatively, if a barrel-less system is used, the desired quantity of solution is programmed into the computer. The barrel-less system is a manifold based, multi-spinneret system. Each spinneret is connected to the manifold, which is fluidly connected to a feed reservoir. The feed rate of the solution is controllable through the use of pressurized fluid which is applied to the reservoir in order to control the rate of dispensation.

Next, the pump is connected to the spinneret assembly and the needle height is adjusted to a predetermined height, optimally 9.00 inches. The positively charged wire is clamped to the needle plate to impose a charge on the solution as it exits the needle tip. The grounded cable is now connected to the substrate (connection plate).

A cooling process is now used in order to ensure that the solvent does not evolve from the polymer until the membrane is formed on the substrate. Failure to perform this cooling step results in the clogging of the needle tip. This volatile evolution can also be reduced using high pressure or a polymer with a lower degree of crystallinity.

The cooling process is performed using a compressed gas to reduce the temperature of the polymer solution inside the spinneret to a temperature of −35 C. This temperature is maintained for the duration of the spinning process.

The DC power supply is also energized to a predetermined value, which for this application is optimally 23 kV. The pump is energized and adjusted to a predetermined flow rate, preferably 0.60 mL/minute. If a syringe barrel system is being used, the pump mechanically moves the barrel through the syringe at a predetermined rate to control flow rate. If a barrel-less system is used, pressure is manipulated to control the flow rate through the spinneret. The particular pump type is less consequential than maintaining a continuous flow rate.

The desired computer program is now run in order to obtain desired sample dimensions. The computer program is similar to a CNC machining operation. An operator defines the X, Y, and Z coordinates, times and rates to the next point. The program can cycle as many time as needed, making a thin layer on each pass, until a desired thickness is achieved, or may achieve a desired thickness in a single pass by adjusting the translation speed accordingly. The desired computer program is now run in order to obtain the appropriate fabric properties, such as thickness, areal density, dimensions. The computer program is a means of storing parameters for a given desired fabric output. Desired areal density and material thickness is determined by customer requirements. For a given polymer flow rate and polymer to solvent ratio, a membrane can be spun to a given areal density based on time of spinning and the size of the spin area.

After the program has run and stopped, the power supply and the pump are turned off and the substrate is removed from the spinning cavity, with the newly electrospun material remaining on the substrate. The material, still attached to the substrate, is preferably placed in a furnace, already preheated to 450° F., for 15 minutes. Notably, fabric properties such as stiffness, thickness, strength, and texture can be altered during this heating procedure, if desired. Additionally, a texture can be imparted onto the fabric by placing the sample on or in between a material with the inverse surface characteristics desired of the fabric. Weights can be added to compress the material during this step, providing more surface area for the intra-fiber cohesion.

If the material is to be calendared, the material, is pressed between rollers having a pressure of 1500 psi. Doing so improves the strength of the material and increases the uniformity of the material thickness and decreases the material porosity and permeability.

The material is then removed from the substrate by gradually pulling on the corners of the screen until the material separates from the screen. The edges of the material are likely to be thinner than the relatively uniform middle portion. These edges are removed and the rest of the material, having a uniform areal density, is cut into customer-desired, application-specific dimensions. For example, a horse shoe shaped 5-10 mm thick piece would be ideal for knee meniscal implants.

The material is now ready to be inspected and packaged. Inspection, at a minimum, tests one or more samples per “run” to determine properties of the material such as thickness, porosity, and aerial density. Again, these variables are application specific and highly selectable.

Process for Making a Reinforced Electrospun Material with a Scrim

Now described is a preferred method of using the electrospinning technology to create an electrospun covering for a scrim. Scrims are used for applications where additional material strength is required. An example of an application is a hernia mesh having anti adhesion properties. For a hernia mesh, a polymer such as PGA, PCL, PDO, HA, hydrogel or mixes of these, is spun directly onto a more standard knitted mesh such as Prolene™ made by Johnson & Johnson. Furthermore, the same technique can be used to spin a polymer directly onto a stent surface. A polymer-based solution is prepared, preferably of a polymer and a solvent, and the solution is spun onto a surface of a fabric scrim or stent. In some cases a priming step is required, as discussed earlier, while other times, wet fibrils are sufficient to bond the membrane directly to the scrim.

A preferred polymer solution for this application is developed by mixing a polymer, preferably polydioxanone (PDO) at 7.5% by mass, with a solvent, preferably HFIP at 92.50% by mass and letting the mixture fully dissolve. Additionally, prior to spinning, the polypropylene scrim must be cleaned such that it accepts the electrospun material. A cleaning solution of 33% butanol and 67% hexane is preferred. Cleaning is accomplished by soaking the polypropylene scrim in the cleaning solution for approximately 30 seconds and allowing the scrim to dry.

In addition to being cleaned, the scrim must also be surface coated or primed. The polymer spinning solution, described above, may be used as the coating solution. Best results are achieved by soaking the scrim in the solution for approximately one minute and removing the scrim therefrom. It is important, for optimal adhesion between the scrim and the electrospun covering, that the surface of the scrim not be allowed to dry before electrospinning commences. Preferably, the priming coat is less than 10 microns thick.

The scrim substrate is placed into the bottom of the spinning cavity, over a grounded plate. The scrim is an electrical insulator so the plate must be well grounded. The needle height is then adjusted to eight inches above the top surface of the scrim.

The motion controller and the computer of the electrospinning device are energized. The MD2 computer program for the motion controller is initialized. Prior to running the program, a predetermined quantity of the solution, preferably 4.0 mL, is transferred into the spinneret. If a barrel system is being used, the piston is inserted into the bore of the spinneret barrel, the barrel assembly is inverted, and the piston is depressed until all the air has been ejected from the barrel. A needle, preferably a 20 gauge needle, is then secured to the end of the barrel.

Alternatively, if a barrel-less system is used, the desired quantity of solution is programmed into the computer. The barrel-less system is a manifold based, multi-spinneret system. Each spinneret is connected to the manifold, which is fluidly connected to a feed reservoir. The feed rate of the solution is controllable through the use of pressurized fluid which is applied to the reservoir in order to control the rate of dispensation.

Next, the pump is connected to the spinneret assembly and the needle height is adjusted to a predetermined height, optimally starting at 8 inches, holding there for 1 minute, and adjusting the height to 12 inches for the remainder of the process. Starting with a needle height of 8 inches for 1 minute provides an initial, wet covering that adheres well to the substrate. Later raising the needle height to 12 inches for the remainder of the process creates an adequately lofty material layer with the desired porosity. The DC power supply is also energized to a predetermined value, which for this application, is optimally 18 kV.

The pump is energized and adjusted to a predetermined flow rate, preferably 0.60 mL/minute. If a syringe barrel system is being used, the pump mechanically moves the barrel through the syringe at a predetermined rate to control flow rate. If a barrel-less system is used, pressure is manipulated to control the flow rate through the spinneret. The particular pump method used is inconsequential as long as a continuous, steady flow rate is maintained.

The desired computer program is now run in order to obtain desired sample dimensions. The computer program is similar to a CNC machining operation. An operator defines the X, Y and Z coordinates, times and rates to the next point. The program can cycle as many time as needed, making a thin layer on each pass, until a desired thickness is achieved, or may achieve a desired thickness in a single pass by adjusting the translation speed accordingly. The desired computer program is now run in order to obtain the appropriate fabric properties, such as thickness, areal density, dimensions. The computer program is a means of storing parameters for a given desired fabric output. Desired areal density and material thickness is determined by customer requirements. For a given polymer flow rate and polymer to solvent ratio, a membrane can be spun to a given areal density based on time of spinning and the size of the spin area.

After the program has run and stopped, the power supply and the pump are turned off and the substrate is removed from the spinning cavity, with the newly electrospun material and scrim remaining on the substrate. The material is allowed to cure before it is removed from the substrate. Preferably, for this application, the material is allowed to cure for at least three hours. The material is then removed from the substrate by gradually pulling on the corners of the screen until the material separates from the screen.

Next the newly formed material is cut into pieces of a predetermined size and shape. The size and shape of the material is determined by customer request or, if packaged based on a use specific application, by intended use.

The material is now ready to be inspected and packaged. Inspection, at a minimum, tests one or more samples per “run” to determine properties of the material such as thickness, porosity, areal density, suture retention, and ball burst. Optimal values for each are determined by customer demands. If acceptable, the other pieces are packed individually into separate containers. Pouches made of a lint-free material such as Tyvek®, made by DuPont®, adequately protect the pieces. Finally, the material and pouches are sterilized.

Process for Making a Textured Electrospun Material with a Scrim

Now described is a preferred method of using the electrospinning technology to create a textured electrospun covering for a scrim. This textured surface produces a more stable membrane using small tack down spots; portions of the fabric that have been locally bonded by an embossed mold. Additionally, the texture improves the ability of the membrane to wick fluids, improves the flexibility of the material, allows the material to drape better, and reduces stiffness. The process is initially identical to the process described above for making an electrospun material with a scrim.

The texturing aspect of this process begins after the scrim is removed from the spinning cavity. Again, the scrim is not removed from the screen. However, before the scrim is cured for three hours, the electrospun membrane is placed on a textured surface mold or rolling mold, and a textured surface imprint is applied to the outer surface of the membrane.

The material is then allowed to cure for three hours, as described above. The rest of the process through packaging and sterilizing remains the same.

Process for Making a Cloth Having a Controlled Drug Release Rate

Now described is a preferred method of using the electrospinning technology of the present invention to control the drug release rate of a drug-eluting object or cloth. By covering the object or cloth with an electrospun covering, having very small interstices, the drug-release kinetics of the object or cloth can be controlled. The manufacturing method is very similar to that of the process for making a reinforced electrospun material with a scrim.

A polymer-based solution is prepared, preferably of a polymer and a solvent, and the solution is spun onto a surface of a drug-containing object such as a fabric, preferably an electrospun fabric, or a stent. In some cases a priming step is required, as discussed earlier, while other times, wet fibrils are sufficient to bond the membrane directly to the scrim.

A preferred polymer solution for this application is developed by mixing a polymer, preferably polydioxanone (PDO) at 7.5% by mass, with a solvent, preferably HFIP at 92.50% by mass and letting the mixture fully dissolve. Additionally, prior to spinning, unless the substrate material is itself electrospun, the drug-containing cloth or object must be cleaned such that it accepts the electrospun material. A cleaning solution of 33% butanol and 67% hexane is preferred. Cleaning is accomplished by soaking the polypropylene scrim in the cleaning solution for approximately 30 seconds and allowing the scrim to dry. In addition to being cleaned, the drug-containing object or cloth should also be surface coated or primed. The polymer spinning solution, described above, may be used as the coating solution. Best results are achieved by soaking the object or cloth in the solution for approximately one minute. It is important, for optimal adhesion between the object or cloth and the electrospun covering, that the surface of the object or cloth not be allowed to dry before electrospinning commences. Preferably, the priming coat is less than 10 microns thick.

The drug-containing object or cloth is placed into the bottom of the spinning cavity, over a grounded plate. If the object is an electrical insulator, the plate must be well grounded. The needle height is then adjusted to eight inches above the top surface of the scrim. If the object is a cloth, the cloth is placed on a grounded substrate.

The motion controller and the computer of the electrospinning device are energized. The MD2 computer program for the motion controller is initialized. Prior to running the program, a predetermined quantity of the solution, preferably 4.0 mL, is transferred into the spinneret. If a barrel system is being used, the piston is inserted into the bore of the spinneret barrel, the barrel assembly is inverted, and the piston is depressed until all the air has been ejected from the barrel. A needle, preferably a 20 gauge needle, is then secured to the end of the barrel.

Alternatively, if a barrel-less system is used, the desired quantity of solution is programmed into the computer. The barrel-less system is a manifold based, multi-spinneret system. Each spinneret is connected to the manifold, which is fluidly connected to a feed reservoir. The feed rate of the solution is controllable through the use of pressurized fluid which is applied to the reservoir in order to control the rate of dispensation.

Next, the pump is connected to the spinneret assembly and the needle height is adjusted to a predetermined height, optimally starting at 8 inches, holding there for 1 minute, and adjusting the height to 12 inches for the remainder of the process. Starting with a needle height of 8 inches for 1 minute provides an initial, wet covering that adheres well to the substrate. Later raising the needle height to 12 inches for the remainder of the process creates an adequately lofty material layer with the desired porosity. The DC power supply is also energized to a predetermined value, which for this application, is optimally 18 kV.

The pump is energized and adjusted to a predetermined flow rate, preferably 0.60 mL/minute. If a syringe barrel system is being used, the pump mechanically moves the barrel through the syringe at a predetermined rate to control flow rate. If a barrel-less system is used, pressure is manipulated to control the flow rate through the spinneret. The particular pump method used is inconsequential as long as a continuous, steady flow rate is maintained.

The desired computer program is now run in order to obtain desired sample dimensions. The computer program is similar to a CNC machining operation. An operator defines the X, Y, and Z coordinates, times and rates to the next point. The program can cycle as many time as needed, making a thin layer on each pass, until a desired thickness is achieved, or may achieve a desired thickness in a single pass by adjusting the translation speed accordingly. The desired computer program is now run in order to obtain the appropriate fabric properties, such as thickness, areal density, dimensions. The computer program is a means of storing parameters for a given desired fabric output. Desired areal density and material thickness is determined by customer requirements. For a given polymer flow rate and polymer to solvent ratio, a membrane can be spun to a given areal density based on time of spinning and the size of the spin area.

After the program has run and stopped, the power supply and the pump are turned off and the substrate is removed from the spinning cavity, with the newly electrospun material and scrim remaining on the substrate. The material is allowed to cure before it is removed from the substrate. Preferably, for this application, the material is allowed to cure for at least three hours. The material is then removed from the substrate by gradually pulling on the corners of the screen until the material separates from the screen.

If the object is a cloth, the cloth is turned over and replaced onto the substrate and the process is repeated so that both sides of the cloth are covered. If the object is three dimensional, the object is manipulated appropriately and the process repeated until a desired amount of the object is covered. Preferably, the object is rotated during the initial covering process.

If the object is a cloth to be used as a drug-eluting bandage, the newly formed material is cut into pieces of a predetermined size and shape. The size and shape of the material is determined by customer request or, if packaged based on a use specific application, by intended use.

The material is now ready to be inspected and packaged. Inspection, at a minimum, tests one or more samples per “run” to determine properties of the material such as thickness, porosity, areal density, suture retention, and ball burst. Optimal values for each are determined by customer demands. If acceptable, the other pieces are packed individually into separate containers. Pouches made of a lint-free material such as Tyvek®, made by DuPont®, adequately protect the pieces. Finally, the material and pouches are sterilized.

Process for Binding a Previously-Spun Material to an Object

Now described is a preferred method of using the electrospinning technology to bind a previously-spun polymer to a substrate such as a stent, scrim, or other object. As demonstrated above, a polymer solution adheres to a substrate when electrospun in a manner that results in wet fibrils contacting the substrate object. It has also been demonstrated that wet spun polymers are particularly adherent to other spun polymers of the same material. Thus, the electrospinning techniques of the present invention are well suited to creating an adhesion layer useable to bind a previously-spun polymer fabric to an object, especially when the adhesion polymer is the same as that of the previously-spun polymer fabric.

A preferred polymer solution for this application is developed by mixing a polymer, preferably the same polymer as was used to make the material that is to be bound to the object with a solvent. Good results have been obtained bonding PET to stainless steel using PET at 12% by mass, with HFIP at 88% by mass and letting the mixture fully dissolve. Good results have also been obtained binding a spun PGA film to knitted PET and polypropylene webs using PGA at 14% by mass and HFIP at 86% by mass.

Prior to spinning, if the substrate is a polypropylene scrim, the scrim must be cleaned such that it accepts the electrospun material. A cleaning solution of 33% butanol and 67% hexane is preferred. Cleaning is accomplished by soaking the polypropylene scrim in the cleaning solution for approximately 30 seconds and allowing the scrim to dry. In addition to being cleaned, the scrim must also be surface coated or primed. The polymer spinning solution, described above, may be used as the coating solution. Best results are achieved by soaking the scrim in the solution for approximately one minute and removing the scrim therefrom. It is important, for optimal adhesion between the scrim and the electrospun covering, that the surface of the scrim not be allowed to dry before electrospinning commences. Preferably, the priming coat is less than 10 microns thick.

The substrate is placed into the bottom of the spinning cavity, over a grounded plate. If the substrate is an electrical insulator, the plate must be well grounded. The needle height is then adjusted to eight inches above the top surface of the substrate.

The motion controller and the computer of the electrospinning device are energized. The MD2 computer program for the motion controller is initialized. Prior to running the program, a predetermined quantity of the solution, preferably 4.0 mL, is transferred into the spinneret. If a barrel system is being used, the piston is inserted into the bore of the spinneret barrel, the barrel assembly is inverted, and the piston is depressed until all the air has been ejected from the barrel. A needle, preferably a 20 gauge needle, is then secured to the end of the barrel.

Alternatively, if a barrel-less system is used, the desired quantity of solution is programmed into the computer. The barrel-less system is a manifold based, multi-spinneret system. Each spinneret is connected to the manifold, which is fluidly connected to a feed reservoir. The feed rate of the solution is controllable through the use of pressurized fluid which is applied to the reservoir in order to control the rate of dispensation.

Next, the pump is connected to the spinneret assembly and the needle height is adjusted to a predetermined height, optimally starting at 8 inches, which will ensure that the substrate is covered with a wet polymer covering. The DC power supply is also energized to a predetermined value, which for this application, is optimally 18 kV.

The pump is energized and adjusted to a predetermined flow rate, preferably 0.60 mL/minute. If a syringe barrel system is being used, the pump mechanically moves the barrel through the syringe at a predetermined rate to control flow rate. If a barrel-less system is used, pressure is manipulated to control the flow rate through the spinneret. The particular pump method used is inconsequential as long as a continuous, steady flow rate is maintained.

The desired computer program is now run in order to obtain desired sample dimensions. The computer program is similar to a CNC machining operation. An operator defines the X, Y and Z coordinates, times and rates to the next point. The program is designed to provide a single, wet covering over the entire substrate.

After the program has run and stopped, the power supply and the pump are turned off and the substrate is removed from the spinning cavity, with the newly electrospun covering remaining on the substrate. The previously-spun material is then wrapped around the substrate before the newly spun covering is allowed to cure.

Process for Covering an Object with a Polymer Layer

Now described is a preferred method of using the electrospinning technology to create an electrospun covering for an object such as a stent. A polymer-based solution is prepared, preferably of a polymer and a solvent, and the solution is spun onto a surface of a fabric scrim or stent. In some cases a priming step is required, as discussed earlier, while other times, wet fibrils are sufficient to bond the membrane directly to the stent.

A preferred polymer solution for this application is developed by mixing a polymer, preferably polydioxanone (PDO) at 7.5% by mass, with a solvent, preferably HFIP at 92.50% by mass and letting the mixture fully dissolve. Additionally, prior to spinning, the stent must be cleaned such that it accepts the electrospun material. A cleaning solution of 33% butanol and 67% hexane is preferred. Cleaning is accomplished by soaking the stent in the cleaning solution for approximately 30 seconds and allowing the stent to dry.

In addition to being cleaned, the stent may also be surface coated or primed. The polymer spinning solution, described above, may be used as the coating solution. Best results are achieved by dipping the stent in the solution and removing the stent therefrom. It is important, for optimal adhesion between the stent and the electrospun covering, that the surface of the stent not be allowed to dry before electrospinning commences. Preferably, the priming coat is less than 10 microns thick.

The stent substrate is placed into the bottom of the spinning cavity, over a grounded plate. The needle height is then adjusted to eight inches above the top surface of the scrim. The motion controller and the computer of the electrospinning device are energized. The MD2 computer program for the motion controller is initialized. Prior to running the program, a predetermined quantity of the solution, preferably 4.0 mL, is transferred into the spinneret. If a barrel system is being used, the piston is inserted into the bore of the spinneret barrel, the barrel assembly is inverted, and the piston is depressed until all the air has been ejected from the barrel. A needle, preferably a 20 gauge needle, is then secured to the end of the barrel.

Alternatively, if a barrel-less system is used, the desired quantity of solution is programmed into the computer. The barrel-less system is a manifold based, multi-spinneret system. Each spinneret is connected to the manifold, which is fluidly connected to a feed reservoir. The feed rate of the solution is controllable through the use of pressurized fluid which is applied to the reservoir in order to control the rate of dispensation.

Next, the pump is connected to the spinneret assembly and the needle height is adjusted to a predetermined height, optimally starting at 8 inches, holding there for 1 minute, and adjusting the height to 12 inches for the remainder of the process. Starting with a needle height of 8 inches for 1 minute provides an initial, wet covering that adheres well to the substrate. Later raising the needle height to 12 inches for the remainder of the process creates an adequately lofty material layer with the desired porosity. The DC power supply is also energized to a predetermined value, which for this application, is optimally 18 kV.

The pump is energized and adjusted to a predetermined flow rate, preferably 0.60 mL/minute. If a syringe barrel system is being used, the pump mechanically moves the barrel through the syringe at a predetermined rate to control flow rate. If a barrel-less system is used, pressure is manipulated to control the flow rate through the spinneret. The particular pump method used is inconsequential as long as a continuous, steady flow rate is maintained.

The desired computer program is now run in order to obtain desired sample dimensions. The computer program is similar to a CNC machining operation. An operator defines the X, Y, and Z coordinates, times and rates to the next point. The program can cycle as many time as needed, making a thin layer on each pass, until a desired thickness is achieved, or may achieve a desired thickness in a single pass by adjusting the translation speed accordingly. The desired computer program is now run in order to obtain the appropriate fabric properties, such as thickness, areal density, dimensions. The computer program is a means of storing parameters for a given desired fabric output. Desired areal density and material thickness is determined by customer requirements. For a given polymer flow rate and polymer to solvent ratio, a membrane can be spun to a given areal density based on time of spinning and the size of the spin area.

After the program has run and stopped, the power supply and the pump are turned off and the stent is removed from the spinning cavity, with the newly electrospun covering remaining on the stent.

Process for Coating an Object with a Polymer

The covered stents just described are optimally suited for forming coated stents that avoid many, if not all, of the problems that the coated stents of the prior art have failed to overcome. The coating method involves heating the covered stent, or other object covered with a fibrous polymer layer, to a temperature at which the electrospun fibrils that span the gaps formed by the braids of the stent separate. When these bridging fibers separate, they tend to contract and collect on the nearest wire. This temperature is maintained until all of the bridging fibrils have separated and collected on their respective wires. The stent is now coated as opposed to being covered.

Notably, the coating retains some of its fibrous qualities. It has been found that raising the temperature, or extending the heating time, or both, effectively reduces the fibrosity of the coating. Reducing the fibrosity of the coating also reduces the porosity of the coating and the size of the interstices between the fibers. If the covered stent is heated long enough or hot enough, the polymer will melt and form a non-fibrous coating on the wires of the stent.

If the object to be coated is temperature sensitive, the same results can be obtained without heat. Instead of heating the covered object, the object is placed in a atmosphere filled with gas from the solvent. By placing the object in this solvent gas chamber, the covering softens and behaves just as though it were being heated. Similarly, the fibrosity of the resulting coating can be affected by the time spent in the chamber and/or the concentration of the solvent gas. The solvent used to form the gas may be the same as that mixed with the polymer to produce the polymer solution for electrospinning.

Process for Electrospinning a Composite Material

Now described is a preferred method of using the electrospinning technology to electrospin a composite material that combines the advantages of two or more polymers into one material. A preferred composite material, described herein, combines the strength of PET with the elasticity of PU. Two polymer-based solution are developed, preferably each of a polymer and a solvent, and the solutions are spun together onto a surface of a substrate.

The first polymer solution for this application is developed by mixing a polymer, preferably PET at 7.50% by mass, with a solvent, preferably HFIP at 92.50% by mass and letting the mixture fully dissolve. The second polymer solution for this application is developed by mixing a polymer, preferably PU at 7.50% by mass, with a solvent, preferably DMAC at 92.50% by mass and letting the second mixture fully dissolve.

Both polymer solutions are then placed into separate spinnerets. However, if the polymers and solvents are mixable, they may be placed into a single spinneret. The needles of the spinnerets are then adjusted to a height of eight inches above the substrate. If the composite material is to be spun onto the surface of a scrim, the scrim is cleaned and primed as described above and the spinnerets are adjusted to eight inches above the surface of the scrim.

The motion controller and the computer of the electrospinning device are energized. The MD2 computer program for the motion controller is initialized. Prior to running the program, a predetermined quantity of the solution, preferably 4.0 mL, is transferred into the spinneret. If a barrel system is being used, the piston is inserted into the bore of the spinneret barrel, the barrel assembly is inverted, and the piston is depressed until all the air has been ejected from the barrel. A needle, preferably a 20 gauge needle, is then secured to the end of the barrel.

Alternatively, if a barrel-less system is used, the desired quantity of solution is programmed into the computer. The barrel-less system is a manifold based, multi-spinneret system. Each spinneret is connected to the manifold, which is fluidly connected to a feed reservoir. The feed rate of the solution is controllable through the use of pressurized fluid which is applied to the reservoir in order to control the rate of dispensation.

Next, the pump is connected to the spinneret assembly and the DC power supply is also energized to a predetermined value, which for this application, is optimally 18 kV. The pump is energized and adjusted to a predetermined flow rate, preferably 0.60 mL/minute. If a syringe barrel system is being used, the pump mechanically moves the barrel through the syringe at a predetermined rate to control flow rate. If a barrel-less system is used, pressure is manipulated to control the flow rate through the spinneret. The particular pump method used is inconsequential as long as a continuous, steady flow rate is maintained.

The desired computer program is now run in order to obtain desired sample dimensions. The computer program is similar to a CNC machining operation. An operator defines the X, Y, and Z coordinates, times and rates to the next point. The program can cycle as many time as needed, making a thin layer on each pass, until a desired thickness is achieved, or may achieve a desired thickness in a single pass by adjusting the translation speed accordingly. The desired computer program is now run in order to obtain the appropriate fabric properties, such as thickness, areal density, dimensions. The computer program is a means of storing parameters for a given desired fabric output. Desired areal density and material thickness is determined by customer requirements. For a given polymer flow rate and polymer to solvent ratio, a membrane can be spun to a given areal density based on time of spinning and the size of the spin area.

After the program has run and stopped, the power supply and the pump are turned off and the substrate is removed from the spinning cavity, with the newly electrospun material and scrim remaining on the substrate. The material is allowed to cure before it is removed from the substrate. Preferably, for this application, the material is allowed to cure for at least three hours. The material is then removed from the substrate by gradually pulling on the corners of the screen until the material separates from the screen.

Next the newly formed material is cut into pieces of a predetermined size and shape. The size and shape of the material is determined by customer request or, if packaged based on a use specific application, by intended use.

The material is now ready to be inspected and packaged. Inspection, at a minimum, tests one or more samples per “run” to determine properties of the material such as thickness, porosity, aerial density, suture retention, and ball burst. Optimal values for each are determined by the application and/or customer demands. If acceptable, the other pieces are packed individually into separate containers. Pouches made of a lint-free material such as Tyvek®, made by DuPont®, adequately protect the pieces. Finally, the material and pouches are sterilized.

Process for Electrospinning a Composite Material for Fuel Cells

Now described is a preferred method of using the electrospinning technology to electrospin a composite material that is optimally suited for use in fuel cells. The preferred composite material, described herein, combines the strength of PET with the electrically filtering properties of Nafion®, made by DuPont®. Two polymer-based solutions are developed, preferably each of a polymer and a solvent and the solutions are spun together onto a surface of a scrim, prepared as described above.

The first polymer solution for this application is developed by mixing a polymer, preferably PET at 7.50% by mass, with a solvent, preferably HFIP at 92.50% by mass and letting the mixture fully dissolve. The second polymer solution for this application is developed by mixing Nafion®, a barrier polymer designed to filter ions, at 7.50% by mass, with a solvent, preferably HFIP, at 92.50% by mass and letting the second mixture fully dissolve. Nafion® is also available in solution form and can be used in this form to achieve acceptable results.

Both polymer solutions are then placed into separate spinnerets. However, if the polymers are mixable, they may be placed into a single spinneret. The needles of the spinnerets are then adjusted to a height of eight inches above the scrim.

The motion controller and the computer of the electrospinning device are energized. The MD2 computer program for the motion controller is initialized. Prior to running the program, a predetermined quantity of the solution, preferably 4.0 mL, is transferred into the spinneret. If a barrel system is being used, the piston is inserted into the bore of the spinneret barrel, the barrel assembly is inverted, and the piston is depressed until all the air has been ejected from the barrel. A needle, preferably a 20 gauge needle, is then secured to the end of the barrel.

Alternatively, if a barrel-less system is used, the desired quantity of solution is programmed into the computer. The barrel-less system is a manifold based, multi-spinneret system. Each spinneret is connected to the manifold, which is fluidly connected to a feed reservoir. The feed rate of the solution is controllable through the use of pressurized fluid which is applied to the reservoir in order to control the rate of dispensation.

Next, the pump is connected to the spinneret assembly and the DC power supply is also energized to a predetermined value, which for this application, is optimally 18 kV. The pump is energized and adjusted to a predetermined flow rate, preferably 0.60 mL/minute. If a syringe barrel system is being used, the pump mechanically moves the barrel through the syringe at a predetermined rate to control flow rate. If a barrel-less system is used, pressure is manipulated to control the flow rate through the spinneret. The particular pump method used is inconsequential as long as a continuous, steady flow rate is maintained.

The desired computer program is now run in order to obtain desired sample dimensions. The computer program is similar to a CNC machining operation. An operator defines the X, Y and Z coordinates, times and rates to the next point. The program can cycle as many time as needed, making a thin layer on each pass, until a desired thickness is achieved, or may achieve a desired thickness in a single pass by adjusting the translation speed accordingly. The desired computer program is now run in order to obtain the appropriate fabric properties, such as thickness, areal density, dimensions. The computer program is a means of storing parameters for a given desired fabric output. Desired areal density and material thickness is determined by customer requirements. For a given polymer flow rate and polymer to solvent ratio, a membrane can be spun to a given areal density based on time of spinning and the size of the spin area.

After the program has run and stopped, the power supply and the pump are turned off and the substrate is removed from the spinning cavity, with the newly electrospun material and scrim remaining on the substrate. The material is allowed to cure before it is removed from the substrate. Preferably, for this application, the material is allowed to cure for at least three hours. The material is then removed from the substrate by gradually pulling on the corners of the screen until the material separates from the screen.

Next the newly formed material is cut into pieces of a predetermined size and shape. The size and shape of the material is determined by customer request or, if packaged based on a use specific application, by intended use.

The material is now ready to be inspected and packaged. Inspection, at a minimum, tests one or more samples per “run” to determine properties of the material such as thickness, porosity, aerial density, suture retention, and ball burst. Optimal values for each are determined by application and/or customer preference. If acceptable, the other pieces are packed individually into separate containers. Pouches made of a lint-free material such as Tyvek®, made by DuPont®, adequately protect the pieces.

Those skilled in the art will further appreciate that the present invention may be embodied in other specific forms without departing from the spirit or central attributes thereof. In that the foregoing description of the present invention discloses only exemplary embodiments thereof, it is to be understood that other variations are contemplated as being within the scope of the present invention. Accordingly, the present invention is not limited in the particular embodiments which have been described in detail therein. Rather, reference should be made to the appended claims as indicative of the scope and content of the present invention.

What is claimed is:

1. A method of controlling the drug release rate of an implantable drug-containing object comprising covering the object with a fibrous fabric having interstices defined between the fibers that are small enough to control the rate at which a drug contained by the object may elute into tissue surrounding the object when the object is implanted.

2. The method of claim 1 wherein covering the object with a fibrous fabric having interstices defined between the fibers that are small enough to control the rate at which a drug contained by the object may elute into tissue surrounding the object when the object is implanted comprises covering the object with a polymer fabric having a plurality of fibrils having diameters that average less than 100 micrometers.

3. The method of claim 1 wherein covering the object with a fibrous fabric having interstices defined between the fibers that are small enough to control the rate at which a drug contained by the object may elute into tissue surrounding the object when the object is implanted comprises covering the object with a polymer fabric having a plurality of fibrils that are intertangled with each other but not woven.

4. A drug-eluting cloth comprising:
an inner layer of fibers of a first average diameter and defining interstices between the fibers;
a therapeutic releasably contained by the inner layer;
an outer layer of fibers of a second average diameter and defining interstices between said fibers that are smaller than the interstices of the inner layer such that the release rate of the therapeutic is controlled by the interstices of the outer layer;
wherein the outer layer is operably attached to and substantially encasing the inner layer.

5. The drug-eluting cloth of claim 4 wherein the fibers of the outer layer comprise electrospun fibrils having an average diameter of less than 100 micrometers.

6. The drug-eluting cloth of claim 4 wherein the fibers of the inner and outer layers comprise electrospun fibrils.

7. The drug-eluting cloth of claim 6 wherein the first average diameter is greater than the second average diameter.

8. A drug eluding cloth of claim 4 wherein the outer layer comprises a first polymer and the inner layer comprises a second polymer different than the first polymer.

9. The drug-eluting cloth of claim 4 wherein the therapeutic releasably contained by the inner layer is disposed in at least a portion of the interstices of the inner layer.

10. The drug-eluting cloth of claim 4 wherein the therapeutic releasably contained by the inner layer is encased in a plurality of microspheres, which are disposed in at least a portion of the interstices of the inner layer.
